(19) United States
(12) Patent Application Publication
(10) Pub. No.: US 2024/0415446 A1
(43) Pub. Date: Dec. 19, 2024

(54) SYSTEM FOR WOUND ANALYSIS

(71) Applicant: Streamlined Forensic Reporting Limited, London (GB)

(72) Inventors: Johann GRUNDLINGH, London (GB); Enrico MARICONTI, London (GB)

(73) Assignee: Streamlined Forensic Reporting Limited, London (GB)

(21) Appl. No.: 18/714,293

(22) PCT Filed: Nov. 30, 2022
(86) PCT No.: PCT/GB2022/053024

§ 371 Date: May 29, 2024

(30) Foreign Application Priority Data
Nov. 30, 2021 (GB) 2117246.5

(51) Int. Cl. A61B 5/00; A61B 5/107; G16H 15/00; G16H 20/00

(52) U.S. Cl. CPC A61B 5/445; A61B 5/1079; A61B 5/7267; G16H 15/00; G16H 20/00

(57) ABSTRACT

A system for classifying wounds is configured to receive an image of a wound, to determine a classification of the wound, and to provide feedback to a user based on the classification of the wound.

### BACKGROUND

This disclosure relates to a system for classifying wounds. In particular, the system receives images as input, from which a classification of a wound is determined. The system is for classifying and optionally further analysing wounds such as surface wounds suffered by a medical patient, or by a victim of crime, for example.

When accidents occur which result in injury, or a crime is committed in which a victim is left with a wound, it is necessary for the wound to be analysed and classified. From a medical perspective, it is important for medical personnel to identify the type of the wound that has been suffered by the patient in order to be able to treat the wound in an appropriate way. For example, a burn must be treated differently to an abrasion (i.e. a graze), or a cut.

In addition to determining the type of the wound suffered, the medic may benefit from identifying further information about the wound, such as the depth of an incised wound, or the severity of a burn, or the age of a bruise, for example. This additional information can be used to determine a method to manage and treat the wound, and may also inform further decisions about whether further intervention is required such as exploratory procedures or further testing on the patient (taking blood samples, for example).

Wound classification is a task that, although not excessively complicated for a doctor, entails a burden in terms of resources allocated as well as the saturation of emergency services due to the patients that may not need any treatment but queue at an emergency department for consultation. Further, where multiple patients require immediate treatment, there is often insufficient capacity of medical staff available to be able to spend time preparing a detailed analysis of the patient's wound for later inclusion in a report; medics must instead work to treat the patients immediately.

Alongside medical analysis and treatment, in the case where the patient has been the victim of a violent crime or an accident such as a road accident, for example, authorities may benefit from determining information about the type of the wound. For example, police investigators may prepare a report on the wound(s) suffered by the victim of the accident or crime. It is common for such a report to be prepared by a medic, based on evidence seen by that medic through an earlier examination, and in line with strict procedures documenting the type of the wound alongside notes and observations concerning the wound. These procedures must comply with local legal requirements so as to be acceptable as evidence in court proceedings, for example, against the perpetrator of the crime. In the context of a crime scene report, it is possible that the person being analysed is deceased.

Further, where an accident has occurred it is common for an insurance claim to be made to compensate the person who has been aggrieved, or to pay for restoration of a vehicle for example. In such cases the party providing insurance benefits from accurate reporting of any injury suffered, and any information about the potential cause of the injury.

In the above cases there is a need to provide accurate evidence-based reporting of details of wounds in an efficient, accurate, and timely manner.

Prior reporting has been compiled by medics treating the patient as their primary care-provider, or otherwise by an independent body engaged for the specific purpose of compiling a report after or during the treatment of the patient. In either case, time and effort is expended accessing the patient and analysing the nature of the medical tests conducted and results obtained, in order to provide an accurate assessment of the available evidence.

However, many medics are not proficient in producing reports containing details of wounds to the level required for legal analysis—by a court, for example. The details that are important to a medic treating a patient do not align strictly with the details that are important to an investigator or judge using the report as evidence to establish the events that led to the wound being received. For this reason, it is common for the reports produced by medics to lack necessary details, and in some cases at the point the report is produced or analysed, it is no longer possible to obtain those details due to the length of time that has passed. For example, the patient may have been treated and so details of the wound, in its original form, can no longer be seen.

Furthermore, it is common for the expression of a wound to change significantly over time. For example, at the time of a road traffic accident when a passenger receives a blow to the head during a collision between vehicles, the visible expression of that wound will differ greatly after two minutes, thirty minutes, two hours, and two days. Bruising may not be immediately visible, but may express shortly after an impact. As the bruise develops, its colouring, size, etc. will change over the coming hours and days. Therefore, at the point of a person beginning to compile a report on the wound suffered by the patient, differing levels of information may be available depending on the time that has passed since the wound was received.

### BRIEF SUMMARY

In one aspect, a system for classifying wounds is configured to receive an image of a wound, to determine a classification of the wound, and to provide feedback to a user based on the classification of the wound.

In another aspect, a method of classifying wounds includes receiving an image of a wound. The method includes, at a first module for classifying a wound, determining a classification of the wound. The method includes providing feedback to a user based on the classification of the wound.

The systems and methods described herein may provide an accurate and consistent analysis of wounds, which is not dependent on the assessment of any single medic or observer. The system provides, in an efficient and repeatable way, classification of wounds based on images that can be obtained under a variety of conditions. In addition to classifying the type of the wound suffered by the patient, the system may determine additional details of the wound such as its depth, size, or the like, and the time that has passed since the wound was received.

The systems and methods described herein seek to reduce or overcome one or more of the deficiencies associated with the prior art.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 is a diagram of the system, embodying the present disclosure;

FIG. 2 is a diagram showing an implementation of the system's core technology, and applications of the system, embodying the present disclosure; and

FIG. 3 is a diagram illustrating hardware components of the system.

### DETAILED DESCRIPTION

The systems and methods describe herein may leverage the use of artificial intelligence, and machine learning, to analyse and classify data relating to wounds.

More particularly, the systems and methods described herein may use Convolutional Neural Networks (CNNs) to determine the classification of wounds.

It should be understood that while in the example set out below we describe the use of CNNs, other types of artificial neural network, and other learning frameworks, may also be suitable for performing the classification task. Support Vector Machines, clustering algorithms (such as K-nearest neighbours) or logistic regression, for example, may be used as an alternative. References herein to a CNN should be read broadly to refer to the use of a trainable learning framework such as those mentioned above. That said, it should be understood that use of a CNN is the preferred learning framework for this task, due to its adaptability and ability to learn details of distinguishing identifiers that can be applied widely across different portions of an image, in different relative lighting conditions, etc., without requiring significant additional pre-processing to remove extraneous detail from the images (compared to the other mentioned algorithms, for example).

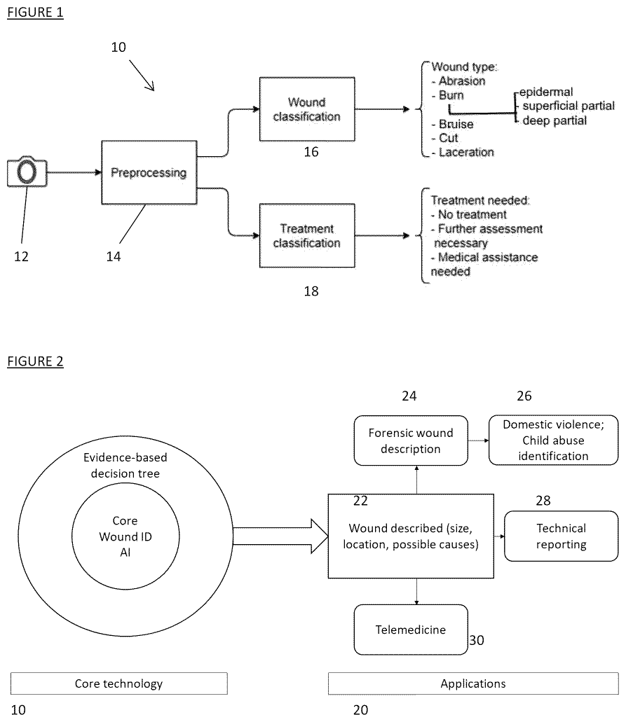

With reference to FIG. 1, the system 100 according to the invention receives data defining an image as an input 12 (either from a camera integrated with the system, or from a remote source), the image containing details of a wound suffered by a person (i.e., a ‘patient’). For example, the image is typically a photograph of the body part of the patient to which the wound has been received. We will refer to this original image as an “image of the wound”; it should be understood that the original image will most likely contain portions that are not of the wound (such as skin surrounding the wound, for example).

In embodiments of the technology, the image of the wound is pre-processed 14 so as to enhance the image for classification, before being processed by the CNN. Pre-processing may include one or more steps of the following: converting between portrait/landscape orientation, resizing the image, centring the wound within the image, rotating the image, altering brightness, altering contrast, cropping the image, applying a preconfigured filter to the image (to emphasise or reduce one or more colour shades within the image), normalising the colour saturation within the image, normalising the brightness levels within the image.

In embodiments of the technology, a calibration step is performed, wherein the image of the wound includes a calibration marker. For example, a white card (or other marker of known colour) may be placed next to the wound (or otherwise held within the field of view of the image) while a photograph is taken. That known marker can then be used to adjust or otherwise scale the lighting or colouring within the photograph (i.e. to ensure that the shading of the white card within the image is adjusted to be white within the image being processed).

In embodiments of the technology, the images processed by the CNN are of a standard size. For example, the size may be 300×300 pixels, or 800×800 pixels. It should be understood that images of lower or greater resolution may be used, subject to the requirements of the system, and the images may be of different aspect ratios. One such requirement may be that if the system is to be deployed on a mobile device (such as installed as an application on a smartphone or tablet computer) then a relatively low resolution such as 300×300 pixels may be appropriate. In this way, the computational processing performed by the CNN is less than would be the case for an image of higher resolution, allowing quicker classification than would otherwise be achieved.

A first module 16 determines the type of wound that can be seen in the received image. This module, referred to as a “wound classifier”, determines whether the wound in the image is one of the following:
an abrasion,
a bruise,
an incised wound,
a laceration,
a burn.

Where it is identified that the wound is a burn, the wound classifier may further determine that the wound is a specific category of burn, for example, without limitation, a superficial partial burn, a deep partial burn, and an epidermal burn.

Where it is identified that the wound is an incised wound (also referred to as an “incision”), the wound classifier may further determine that the wound is a stab or a cut.

The wound classifier is configured during the training of the CNN to identify and distinguish images of wounds according to learned properties of the images. For example, the following properties may establish the classifications of wounds based on their visible appearances:

Abrasion: Superficial scuffing injury with a directional component to the scuff, caused by tangential motion (i.e. relative to a surface). These may be caused by blunt or sharp forces/impacts.

Laceration: Tearing or splitting of the skin due to crushing or shearing forces. Typically, these lacerations display irregular or jagged edges, although the edges are sometimes clean if over a bony surface. Commonly lacerations display abraded and bruised edges of the wound with tissue bridges, and are common over bony prominences.

Bruise: Extravascular collection of blood that has leaked from a blood vessel damaged by a mechanical impact. The colour of the bruise may be of help to determine the age, and its appearance may provide information about a weapon or object used to cause an attack in an assault, for example. The colouring of a bruise may appear or develop further one or more days after the injury is caused.

Incised wounds: These include cuts (having a length longer than its depth), and stabs (having a depth greater than its length). The edges of the wound are usually free of damage.

Burns: These include wounds caused by thermal, chemical, friction, electric, radiation or radioactivity injury to the various layers of the skin. Burns lead to inflammation, damage and/or loss of skin and damage to the various skin layers and underlying tissue. Within this category, epidermal burns commonly present with redness of the skin and potentially with mild swelling in the region of the burn. Superficial partial burns are typically characterised by the formation of blisters and/or red blotchy skin presenting with swelling in the region of the burn. Deep partial burns usually present with blotchy red skin, or waxen skin (i.e. smooth and pale) with a white or grey shade and usually without blister formation.

In embodiments of the technology, the system provides two modules that are each trained to solve a different problem. In addition to the first module, a second module 18 determines whether the wound shown in the image requires professional medical treatment. This second module is referred to as a “treatment classifier”. The treatment classifier may receive the image as input directly, or may receive the classified image as input (i.e. provided with the classified wound type from the wound classifier). The treatment classifier determines whether no professional treatment is needed, whether further human assessment is necessary, or whether medical assistance is needed.

In addition, the treatment classifier may determine that, where medical assistance at a medical centre is needed, the immediacy of the required treatment. For example, it may be determined that the wound is potentially life-threatening requiring urgent attention. Or, it may be determined that while the wound does require medical assistance, the wound is of a type that does not require urgent treatment.

Such a treatment classifier, when integrated into an application on a smartphone, for example, allows the user to receive a swift first assessment of the wound as soon as the wound is inflicted, or as soon as the patient is observed. Moreover, in the case of wounds that do not need urgent treatment by a medical professional, it is possible to give advice to the patient according to the classification of wound determined by the wound classifier.

In embodiments of the technology, the system provides additional information to the user alongside the determination of the type of the wound and/or the requirement for treatment. For example, an opportunity for further assessment through a telephone call or a video call (which may be provided to the user, integrated within the application), allows trained medics such as doctors or nurses to assess the wound and provide additional advice. As an alternative to a link or contact details being provided by the application, this might be achieved quickly through the user making a telephone call to a known health service contact number when the treatment classifier provides a determination that further human assessment is needed.

In embodiments of the technology, the system may provide information to a user regarding suitable steps for treating the wound, or immediate actions for cleaning the wound or preventing blood loss from the wound, for example. This information may be determined based on the classification of the wound and/or the probabilities associated with the wound belonging to each category. For example, if the most likely wound classification is a bruise, but there is also a not insignificant probability that the wound might be a burn, then the system may recommend treatments for treating at least a bruise, and potentially also for treating a burn. If any treatment for a bruise would be strictly incompatible with treating a burn, the system may exclude such treatment steps from those displayed to the user. This is to prevent inappropriate treatment steps being taken which might make the wound worse, or lead to complications, should the original classification of a bruise be incorrect.

For any of the above determinations made by either the wound classifier, or the treatment classifier, or both, a confidence parameter may be assigned to the determined classification. For example, the wound classifier may determine that the wound shown in the image is of an abrasion with a determined confidence of 83%. In that case, the wound classifier may return multiple potential classifications each associated with a confidence parameter. For example, the wound classifier may return the determination of classified wound type as:
an abrasion: 83%,
a bruise: 3%,
an incised wound: 1%,
a laceration: 1%,
a burn: 12%.

The multiple potential classifications and associated confidence parameters may be displayed to a user via an interface of a device operating the system, and/or may be incorporated into a patient record or report relating to the wound. In this way, a level of certainty in the classification can be gauged either by the system itself for use in interpreting the results and/or for inclusion in a report generated by the system, or by a person using the system in a medical or reporting capacity.

Based on the determination of confidence parameters associated with one or more of the classifications of the wound, a system presenting feedback of treatment steps to a user may exclude any treatment steps that are incompatible with any of the classifications returning a confidence parameter over a certain threshold. With a confidence parameter of 12% for the classification of the wound being a burn (as in the above example), the system would not display treatments incompatible with burns if the 12% exceeds a threshold set at 5%, for example.

The two modules may in examples comprise CNNs providing six feature learning layers and a further classification layer. The feature learning layers may each have different descent sizes, from 1024 to 32 neurons, for example. The CNNs are structured as is known in the art. The feature learning layers include layers for performing convolution, and subsequently layers for pooling. In the example described here, three pairs of layers are provided, each pair comprising a convolution layer followed by a pooling layer. Subsequently, a softmax function activation function is used to output the classifications (with associated relative probabilities, where required). In other embodiments, a sigmoid activation function may be used.

In embodiments of the system, the CNNs used in the first and second modules may be trained using up to 30 epochs (i.e. the training images are passed through the network up to 30 times in order to update the weights within the network) using a batch size (i.e. number of labelled training images) equal to 64 to find the optimum while minimising the risk of overfitting to the specific training data. It should be understood that larger datasets may be used for training, and that different numbers of epochs for training the CNNs may be employed.

The training images are provided to the CNN with labels of the correct classifications associated with the wounds visible in the images. The classifications may be input by a medical professional that has reviewed the images and determined the correct classifications. The training images may be supplemented by additional training data comprising images that have been processed using pre-processing techniques as discussed above. For example, the colouring of the images may be altered, the position of the wounds within the images may be moved, and the images may be resized and/or rotated or flipped (to provide a mirror image), for example.

The training images may comprise images of the same wound taken at different time intervals. For example, images of a bruise may be taken over time intervals (such as every four hours, every ten hours, or every day) for example, to supplement the data set. The images may be supplemented with metadata concerning the age of the wound and/or other details of the wound as determined by the medic classifying the images for training purposes. In this way, the CNN learns to associate properties of the images with metadata such as the age of the wound and/or other features of the wound.

In embodiments, the system determines additional parameters associated with the wound alongside its classification. Such parameters may include the size of the wound (as in surface area on the skin, or the depth of an incision, for example), the colouring or relative colouring of a bruise to the surrounding skin, an estimated age of the wound and/or a location of the wound on the body of the patient.

In embodiments of the system, multiple images of the patient's wound may be taken, over multiple time intervals. A first image may be taken, and analysed by the wound classifier to determine a wound classification. A subsequent image may be taken at a second time, and processed by the system. At each classification, parameters of the wound may be determined, including the size of the wound. For incised wounds, the depth of the wound may be determined. For bruises and burns in particular (but also for other types of wound), an age of the wound may be determined. The age of a bruise may be determined according to the size, colour, and pattern of the bruise for example. In embodiments of the technology, the system may compare multiple images taken at different times, and may use information from earlier wound determination steps to inform later wound classification steps, so as to ensure consistency of the data provided. For example, information may be determined based on the spread of the wound between successive images, or the rate of apparent healing of the wound.

With reference to FIG. 2, in embodiments of the system 10 providing the core technology of identifying a wound, the result of the classification determined by the system is subsequently analysed using a decision tree structure to determine how to present the information to a user. In the various applications 20 described below, in which reporting or feedback is provided by the system 10, the system provides a feedback module. The feedback module generally provides a description of the wound 22, based on application of a suitable evidence-based decision tree. The decision tree may select an appropriate feedback output based on the classification type, associated certainty of the classification, severity of the wound, requirement for medical assistance, and other factors as discussed above.

The applications of the system 10 include a technical reporting system 28, in which an accurate description of a wound is provided 22. The description of the wound may include the classification alongside details such as the size of the wound, location of the wound on the patient, and possible causes of the wound—which information may be determined in part from the decision tree analysis. This information is subsequently compiled in a technical report on the wound, which may be provided to law enforcement agencies for assisting a criminal prosecution for example, and/or to providers of insurance policies or those investigating an insurance claim. Such reports may include the original images taken of the wound, and may also include notes provided by a medic treating the patient.

In some embodiments of the described technology, the wound classifier may be trained to output a classification not only of a wound type, but also within that classification a subcategory associated with a potential cause of the wound. So, for example, a burn may be further subcategorised not only by the ‘type’ of the burn as described above, but by the probable cause of the burn based on any visual indicators present in the image. Chemical burns may differ significantly in appearance from burns caused by contact with a flame or contact with steam, for example.

In embodiments of the technology the system 10 may be integrated into an image-sharing platform or application, or into a social media platform in which images are posted and shared by users. In this way the system may be used to identify symptoms of domestic violence or child abuse 26, for example. The system may analyse images of people posted online, to determine whether the images include wounds. For example the system may determine a forensic wound description 24 identifying that a photo posted on a website shows a person having bruising on their body. The system may alert the platform provider, or a third party, of the apparent bruising or other wounds determined to be inflicted on the subject of the photograph. The referral may be to a medical provider or to a law enforcement agency, or to an anti-abuse or child protection agency, for example.

In embodiments of the technology, the system may monitor images posted to a user's account over a period of time, to determine whether a pattern of wounds is established. For example, if images are posted on an account that illustrate an isolated occurrence of a wound—or that a series of images over a short period of time show a wound is present—then it is possible/likely that those images all illustrate the same wound. If that wound is a bruise, for example, then a single episode of bruising may simply be explained by isolated accident. In contrast, a persistent or repeating pattern of bruising may be indicative of a person having an underlying health condition, or that the person may be subject to abusive or otherwise violent behaviour.

A system according to embodiments may provide feedback to the user associated with the account on the file-sharing or social media platform, such feedback including one or more of information about the identified wound such as treatment advice; contact details for seeking medical attention; and contact details for reporting abusive behaviour.

As a further application of the system 10, the feedback of the system is used in provision of telemedicine (i.e. remote analysis of a patient). The system 10 may provide a report as outlined above, via an application on a mobile device or via a website, for consideration by a medical professional.

While use of the system as an application on a mobile device is envisaged, the system may also be deployed on a laptop or personal computer. Computational aspects of the system such as the pre-processing of images and/or classification via the CNN, may be performed locally on the device with which the user interacts or alternatively performed on processors provided remotely in the cloud on a server or via a distributed system of servers. Subsequently, the outcome of the analysis may be made available over the internet or via the application, for example, or may be transmitted to a third party device (such as via email or SMS, for example, where a recipient's contact email address or contact telephone number is provided).

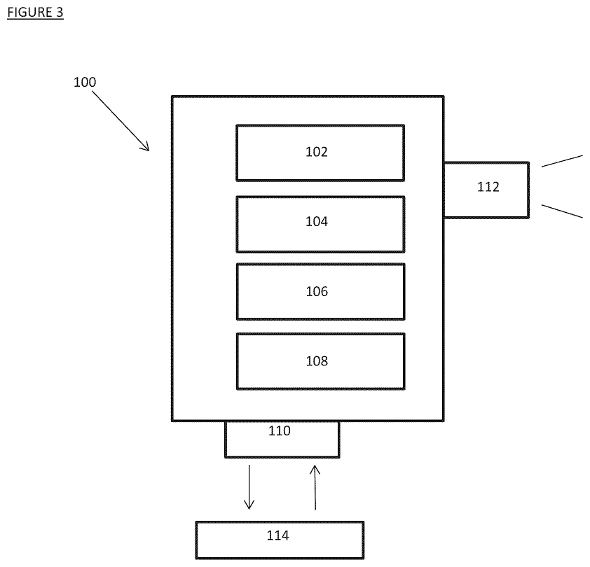

With reference to FIG. 3, a system 100 suitable for carrying out the steps described herein provides a device having one or more processors 102, associated memory 104, and has access to one or more storage devices 106. The system further provides at least a display 108 and/or a communication device 110, so as to provide visual feedback to a user and/or communicate feedback to another device 114. In embodiments of the technology, the system is implemented via smart phone or a similar portable device, providing a camera 112 for taking images (of a wound, for example), a processor 102 for executing instructions for carrying out the methods and processes described herein, and a memory 104 and storage 106. The device may display the results of the computations carried out via its integral display device 108, or may communicate the results remotely to a third party, for example. In some embodiments, the device may communicate the image taken via the camera 112, for processing remotely, and may then receive the result of the classification and/or feedback regarding the classification, from the remote device 114.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Although certain example embodiments of the invention have been described, the scope of the appended claims is not intended to be limited solely to these embodiments. The claims are to be construed literally, purposively, and/or to encompass equivalents.

Representative features are set out in the following clauses, which stand alone or may be combined, in any combination, with one or more features disclosed in the text and/or drawings of the specification.

According to a preferred embodiment of the invention a system for classifying wounds is configured to receive an image of a wound, to determine a classification of the wound, and to provide feedback to a user based on the classification of the wound. The classification of the wound may be one of an abrasion, a bruise, an incised wound, a laceration, and a burn; the system may classify the wound as comprising a plurality of wounds, each of which the system may then proceed to classify. The system may further determine that a wound classified as a burn is one of a specific category of burns, including at least one of a superficial partial burn, a deep partial burn, and an epidermal burn.

The system may include a first module for classifying a wound, and a second module for determining a type of treatment associated with the wound. The treatment may be classified as at least one of no professional treatment is needed, further human assessment is necessary, or medical assistance is needed.

The determination of the classification of the wound may further include determining a confidence parameter associated with the determined wound classification.

The determination of the classification of the wound may include a determination of multiple confidence parameters associated with the wound belonging to each of multiple respective wound classifications.

The determination of the classification of the wound may be made by a convolutional neural network receiving the image of the wound as input.

The image of the wound may be subject to one or more pre-processing steps prior to being input to the convolutional neural network, the pre-processing step(s) being chosen from: converting between portrait/landscape orientation, resizing the image, centring the wound within the image, rotating the image, altering brightness, altering contrast, cropping the image, applying a preconfigured filter to the image, normalising the colour saturation within the image, normalising the brightness levels within the image.

The convolutional neural network may implement a softmax activation function.

The determination of the classification of the wound may include determining one or parameters of the wound, including at least one of: a size of the surface area of the wound; a depth of the wound; a colouring or relative colouring of the wound; an estimated age of the wound; and/or a location of the wound on the body of the patient.

The feedback provided by the system may include at least one of: a report containing the determined classification of the wound; advice concerning treatment of the wound; and/or a report containing a possible cause of the wound.

The system may receive as input an image hosted on a file-sharing or social media platform, and the user may be either a person associated with the image, or a person associated with moderation of the platform, and the feedback provided to the user comprises alerting the user to the identification of the wound.

The feedback provided to the user may include at least one of:
information about the identified wound such as treatment advice;
contact details for seeking medical attention;
contact details for reporting abusive behaviour.

According to another preferred embodiment, a computer-implemented method of classifying wounds includes receiving an image of a wound; at a first module for classifying a wound, determining a classification of the wound; and providing feedback to a user based on the classification of the wound. Receiving an image of a wound may include taking a photograph of the wound using a camera associated with the system.

Determining the classification of the wound may include classifying the wound as one of: an abrasion, a bruise, an incised wound, a laceration, and a burn.

Determining that the wound is classified as a burn may include determining that that the wound is one of a specific category of burns, including at least one of: a superficial partial burn, a deep partial burn, and an epidermal burn.

The method may further include a step of, at a second module for classifying a treatment, determining a type of treatment associated with the wound. Determining a type of treatment may include determining one of: no professional treatment is needed, further human assessment is necessary, or medical assistance is needed.

The method may further include determining a confidence parameter associated with the determined wound classification.

Determining a confidence parameter may include determining multiple confidence parameters associated with the wound belonging to each of multiple respective wound classifications.

The method may further include performing one or more pre-processing steps on the image prior to the step of determining a classification of the wound, the pre-processing step(s) being chosen from: converting between portrait/landscape orientation, resizing the image, centring the wound within the image, rotating the image, altering brightness, altering contrast, cropping the image, applying a preconfigured filter to the image, normalising the colour saturation within the image, normalising the brightness levels within the image.

Determining the classification of the wound may include determining one or parameters of the wound, including at least one of: a size of the surface area of the wound; a depth of the wound; a colouring or relative colouring of the wound; an estimated age of the wound; a location of the wound on the body of the patient

Providing feedback may include communicating at least one of: a report containing the determined classification of the wound; advice concerning treatment of the wound; a report containing a possible cause of the wound.

Receiving an image may include receiving an image hosted on a file-sharing or social media platform, and wherein the user is either a person associated with the image, or a person associated with moderation of the platform, and providing feedback includes alerting the user to the identification of the wound.

Providing feedback may include communicating at least one of: information about the identified wound such as treatment advice; contact details for seeking medical attention; contact details for reporting abusive behaviour.

In some embodiments, the system 100 includes non-transitory, computer-readable medium comprising computer program instructions tangibly stored on the non-transitory computer-readable medium, wherein the instructions are executable by at least one processor to perform each of the steps of the methods described above.

It should be understood that the systems described above may provide multiple ones of any or each of those components and these components may be provided on either a standalone machine or, in some embodiments, on multiple machines in a distributed system. The phrases ‘in one embodiment,’ ‘in another embodiment,’ and the like, generally mean that the particular feature, structure, step, or characteristic following the phrase is included in at least one embodiment of the present disclosure and may be included in more than one embodiment of the present disclosure. Such phrases may, but do not necessarily, refer to the same embodiment. However, the scope of protection is defined by the appended claims; the embodiments mentioned herein provide examples.

The terms “A or B”, “at least one of A or/and B”, “at least one of A and B”, “at least one of A or B”, or “one or more of A or/and B” used in the various embodiments of the present disclosure include any and all combinations of words enumerated with it. For example, “A or B”, “at least one of A and B” or “at least one of A or B” may mean (1) including at least one A, (2) including at least one B, (3) including either A or B, or (4) including both at least one A and at least one B.

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

Although terms such as “optimize” and “optimal” may be used herein, in practice, embodiments of the present invention may include methods which produce outputs that are not optimal, or which are not known to be optimal, but which nevertheless are useful. For example, embodiments of the present invention may produce an output which approximates an optimal solution, within some degree of error. As a result, terms herein such as “optimize” and “optimal” should be understood to refer not only to processes which produce optimal outputs, but also processes which produce outputs that approximate an optimal solution, within some degree of error.

The systems and methods described above may be implemented as a method, apparatus, or article of manufacture using programming and/or engineering techniques to produce software, firmware, hardware, or any combination thereof. The techniques described above may be implemented in one or more computer programs executing on a programmable computer including a processor, a storage medium readable by the processor (including, for example, volatile and non-volatile memory and/or storage elements), at least one input device, and at least one output device. Program code may be applied to input entered using the input device to perform the functions described and to generate output. The output may be provided to one or more output devices.

Each computer program within the scope of the claims below may be implemented in any programming language, such as assembly language, machine language, a high-level procedural programming language, or an object-oriented programming language. The programming language may, for example, be LISP, PROLOG, PERL, C, C++, C#, JAVA, Python, Rust, Go, or any compiled or interpreted programming language.

Each such computer program may be implemented in a computer program product tangibly embodied in a machine-readable storage device for execution by a computer processor. Method steps may be performed by a computer processor executing a program tangibly embodied on a computer-readable medium to perform functions of the methods and systems described herein by operating on input and generating output. Suitable processors include, by way of example, both general and special purpose microprocessors. Generally, the processor receives instructions and data from a read-only memory and/or a random access memory. Storage devices suitable for tangibly embodying computer program instructions include, for example, all forms of computer-readable devices, firmware, programmable logic, hardware (e.g., integrated circuit chip; electronic devices; a computer-readable non-volatile storage unit; non-volatile memory, such as semiconductor memory devices, including EPROM, EEPROM, and flash memory devices; magnetic disks such as internal hard disks and removable disks; magneto-optical disks; and CD-ROMs). Any of the foregoing may be supplemented by, or incorporated in, specially-designed ASICs (application-specific integrated circuits) or FPGAs (Field-Programmable Gate Arrays). A computer can generally also receive programs and data from a storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium. A computer may also receive programs and data (including, for example, instructions for storage on non-transitory computer-readable media) from a second computer providing access to the programs via a network transmission line, wireless transmission media, signals propagating through space, radio waves, infrared signals, etc.

Having described certain embodiments of methods and systems for classifying wounds, it will be apparent to one of skill in the art that other embodiments incorporating the concepts of the disclosure may be used. Therefore, the disclosure should not be limited to certain embodiments, but rather should be limited only by the spirit and scope of the following claims.

1. A system for classifying wounds, the system being configured to receive an image of a wound, to determine a classification of the wound, and to provide feedback to a user based on the classification of the wound.

2. A system according to claim 1, wherein the feedback provided by the system includes at least one of:
advice concerning treatment of the wound;
a report containing a possible cause of the wound;
a report containing the determined classification of the wound.

3. A system according to claim 1, including a first module for classifying a wound, and a second module for determining a type of treatment associated with the wound.

4. A system according to claim 3, wherein the treatment is classified as at least one of:
no professional treatment is needed,
further human assessment is necessary, or
medical assistance is needed.

5. A system according to claim 1, wherein the determination of the classification of the wound further includes determining a confidence parameter associated with the determined wound classification.

6. A system according to claim 5, wherein the determination of the classification of the wound includes a determination of multiple confidence parameters associated with the wound belonging to each of multiple respective wound classifications.

7. A system according to claim 6, in which the feedback provided by the system includes advice concerning treatment of the wound, configured such that if a confidence parameter associated with any one of the potential classifications exceeds a threshold, treatment options that are incompatible with that respective classification are not provided in the feedback to the user.

8. A system according to claim 7, configured such where a first classification is determined by the system to have a first confidence parameter, and a second classification is determined to have a second confidence parameter that is less than the first, a treatment that is suitable for treating a wound of the first classification but is incompatible with a wound of the second classification is omitted from the feedback provided to the user where the second confidence parameter exceeds the threshold.

9. A system according to claim 1, wherein the system receives as input an image hosted on a file-sharing or social media platform, and wherein the user is either a person associated with the image, or a person associated with moderation of the platform, and the feedback provided to the user comprises alerting the user to the identification of the wound.

10. A system according to claim 9, wherein the feedback provided to the user includes at least one of:
information about the identified wound such as treatment advice;
contact details for seeking medical attention;
contact details for reporting abusive behaviour.

11. A system according to claim 1, wherein the classification of the wound is one of:
an abrasion,
a bruise,
an incised wound,
a laceration,
a burn.

12. A system according to claim 11, wherein the system further determines that a wound classified as a burn is one of a specific category of burns, including at least one of:
a superficial partial burn,
a deep partial burn,
an epidermal burn.

13. A system according to claim 1, wherein the determination of the classification of the wound is made by a convolutional neural network receiving the image of the wound as input.

14. A system according to claim 13, wherein the image of the wound is subject to one or more pre-processing steps prior to being input to the convolutional neural network, the pre-processing step(s) being chosen from: converting between portrait/landscape orientation, resizing the image, centring the wound within the image, rotating the image, altering brightness, altering contrast, cropping the image, applying a preconfigured filter to the image, normalising the colour saturation within the image, normalising the brightness levels within the image.

15. (canceled)

16. A system according to claim 1, wherein the determination of the classification of the wound includes determining one or more parameters of the wound, including at least one of:
a size of the surface area of the wound;
a depth of the wound;
a colouring or relative colouring of the wound;
an estimated age of the wound;
a location of the wound on the body of the patient.

17. A computer-implemented method of classifying wounds, comprising the steps of:
receiving an image of a wound,
at a first module for classifying a wound, determining a classification of the wound, and
providing feedback to a user based on the classification of the wound.

18. A computer-implemented method according to claim 17, wherein receiving an image of a wound includes taking a photograph of the wound using a camera associated with the system.

19-20. (canceled)

21. A computer-implemented method according to claim 17, further including a step of, at a second module for classifying a treatment, determining a type of treatment associated with the wound.

22. A computer-implemented method according to claim 21, wherein determining a type of treatment includes determining one of:
no professional treatment is needed,
further human assessment is necessary, or
medical assistance is needed.

23. A computer-implemented method according to claim 17, further including determining a confidence parameter associated with the determined wound classification.

24. A computer-implemented method according to claim 23, wherein determining a confidence parameter includes determining multiple confidence parameters associated with the wound belonging to each of multiple respective wound classifications.

25. A computer-implemented method according to claim 24, wherein providing feedback includes providing advice concerning treatment of the wound, wherein if a confidence parameter associated with any one of the potential classifications exceeds a threshold, treatment options that are incompatible with that respective classification are not provided in the feedback to the user.

26. A computer-implemented method according to claim 25, including determining that a first classification has a first confidence parameter, and that a second classification has a second confidence parameter that is less than the first, and providing feedback to a user that omits treatments that are suitable for treating a wound of the first classification but are incompatible with a wound of the second classification where the second confidence parameter exceeds the threshold.

27-28. (canceled)

29. A computer-implemented method according to claim 17, wherein providing feedback includes communicating at least one of:
a report containing the determined classification of the wound;
advice concerning treatment of the wound;
a report containing a possible cause of the wound.

30-31. (canceled)
